(19) United States
(12) Patent Application Publication
(10) Pub. No.: US 2020/0222828 A1
(43) Pub. Date: Jul. 16, 2020

(54) SHORT PATH DISTILLATION IN VACUUM FOR ENRICHING NATURAL SUBSTANCES

(71) Applicant: BIONORICA ETHICS GMBH, Neumarkt (DE)

(72) Inventors: Andreas RUTZ, Dornhausen (DE); Michael ENGLERT, Nürnberg (DE)

(21) Appl. No.: 16/651,267

(22) PCT Filed: Oct. 1, 2018
(86) PCT No.: PCT/EP2018/076699

§ 371 Date: Mar. 26, 2020

(30) Foreign Application Priority Data
Sep. 30, 2017 (EP) 17194270.9

(51) Int. Cl. B01D 3/10; A61K 9/00; A61K 31/352

(52) U.S. Cl. CPC B01D 3/10; A61K 2236/39; A61K 31/352; A61K 9/0073

(57) ABSTRACT

The present invention relates to a method for producing, obtaining and enriching dronabinol (Δ9-THC) as well as natural substances from plant extracts.

The present invention relates to a method for producing, obtaining and enriching dronabinol (Δ9-THC) as well as natural substances from plant extracts.

Together with the genus Humulus (hops), Cannabis (hemp) belongs to the Cannabaceae family, whereby, however, Humulus does not contain any cannabinoids. Within the genus Cannabis, there is a botanical and chemotaxonomic differentiation, namely in the species Cannabis sativa Linnaeus, Cannabis indica Lam. and Cannabis ruderalis or in the “species complex” Cannabis sativa L., which consists of the sub species Cannabis sativa ssp. sativa and ssp. indica. Cannabis is furthermore differentiated into drug hemp and fiber hemp, whereby the differentiation is based on the quantitative ratios of the primary cannabinoids cannabidiol (CBD) and Δ⁹-tetrahydrocannabinol (Δ⁹-THC) (INN: dronabinol). Fiber hemp (also: industrial hemp) is primarily used to industrially obtain fiber and may not exceed a Δ⁹-THC content of 0.2% (e.g. Germany, among others), while the drug type can have a Δ⁹-THC content of approx. 5-35% (marijuana, hashish). Cannabis sativa L. contains over 400 different ingredients, of which more than 60 compounds belong to the cannabinoid class. The most important cannabinoids are shown in the following:

Cannabigerol-type (CBG): cannabigerol ((E)-CBG-C₅), cannabigerol monomethyl ether ((E)-CBGM-C₅ A), cannabinerolic acid A ((Z)-CBGA-C₅ A), cannabigerovarin ((E)-CBGV-C₃), cannabigerolic acid A ((E)-CBGA-C₅ A), cannabigerolic acid A monomethyl ether ((E)-CBGAM-C₅ A), cannabigerovaric acid A ((E)-CBGVA-C₃ A); Cannabichromene-type (CBC): cannabichromene (CBC—C₅), cannabichromenic acid A (CBCA—C₅ A), cannabichromevarin (CBCV—C₃), cannabichromevarinic acid A (CBCVA-C3 A); Cannabidiol-type (CBD): Cannabidiol (CBD-C₅), cannabidiol monomethyl ether (CBDM-C₅), cannabidiol-C4 (CBD-C₄), cannabidivarin (CBDV-C₃), cannabidiorcol (CBD-C₁), cannabidiolic acid (CBDA-C₅), cannabidivarinic acid (CBDVA-C₃); Cannabinodiol-type (CBND): Cannabinodiol (CBND-C₅), cannabinodivarin (CBND-C₃);

Tetrahydrocannabinol-type (THC): Δ9-Tetrahydrocannabinol (Δ9-THC—C₅), Δ9-tetrahydrocannabinol-C4 (Δ9-THC—C₄), Δ9-tetrahydrocannabivarin (Δ9-THCV—C3), Δ9-tetrahydrocannabiorcol (Δ9-THCO—C₁), Δ9-tetrahydrocannabinolic acid (Δ9-THCA—C₅ A), Δ9-tetrahydrocannabinolic acid B (Δ9-THCA—C₅ B), Δ9-tetrahydrocannabinolic acid-C4 (Δ9-THCA—C₄ A and/or B), Δ9-tetrahydrocannabivarinic acid A (Δ9-THCVA-C₃ A), Δ9-tetrahydrocannabiorcolic acid (Δ9-THCOA-C₁ A and/or B), (−)-A8-trans-(6aR,10aR)-A8-tetrahydrocannabinol (A8-THC—C₅), (−)-A8-trans-(6aR,10aR)-tetrahydrocannabinolic acid A (A8-THCA—C₅ A); (−)-(6aS,10aR)-Δ9-tetrahydrocannabinol ((−)-cis-Δ9-THC—C₅); Cannabinol-type (CBN): Cannabinol CBN—C₅, cannabinol-C4 (CBN—C₄), cannabivarin (CBN—C₃), cannabinol-C2 (CBN—C₂), cannabiorcol (CBN—C₁), cannabinolic acid A (CBNA-C₅ A), cannabinol methylether (CBNM-C₅)

Cannabitriol-type (CBT): (−)-(9R,10R)-trans-cannabitriol ((−)-trans-CBT-C₅), (+)-(9S,10S)-cannabitriol ((+)-trans-CBT-C₅), (±)-(9R,10S/9S,10R)-cannabitriol ((±)-cis-CBT-C₅), (−)-(9R,10R)-trans[10-O-ethyl-cannabitriol] ((−)-trans-CBT-OEt-C₅), (±)-(9R,10R/9S,10S)-cannabitriol-C₃ ((+)-trans-CBT-C₃), 8,9-dihydroxy-A6a (10a) tetrahydrocannabinol (8,9-Di-OH—CBT-C₅), cannabidiolic acid A (CBDA-C₅ 9-OH—CBT-C5 ester), (−)-(6aR,9S,10S,10aR)-9,10-dihydroxy-hexahydrocannabinol, cannabiripsol cannabiripsol-C5, (−)-6a, 7,10a-trihydroxy-Δ9-tetrahydrocannabinol ((−)-cannabitetrol), l0-oxo-A6a (10a) tetrahydrocannabinol (OTHC);

Cannabielsoin-type (CBE): (5aS,6S, 9R, 9aR)-C₅-cannabielsoin (CBE-C₅), (5aS,6S,9R,9aR)-C₃-cannabielsoin (CBE-C3), (5aS,6S,9R,9aR)-cannabielsoinic acid A (CBEA-C₅ A), (5aS,6S,9R,9aR)-cannabielsoinic acid B (CBEA-C₅ B), (5aS,6S,9R,9aR)-C3-cannabielsoinic acid B (CBEA-C₃ B), cannabiglendol-C3 (OH-iso-HHCV—C₃), dehydrocannabifuran (DCBF—C₅), cannabifuran (CBF—C₅);

Isocannabinoids: (−)-A7-trans-(1R,3R,6R)-isotetrahydrocannabinol, (±)-A7-1,2-cis-(1R,3R,6S/1S,3S,6R)-isotetrahydrocannabivarin, (−)-A7-trans-(1R,3R,6R)-isotetrahydrocannabivarin;

Cannabicyclol-type (CBL): (+)-(1aS,3aR,8bR,8cR)-cannabicyclol (CBL-C5), (±)-(1aS,3aR,8bR,8cR)-cannabicyclolic acid A (CBLA-C₅ A), (±)-(1aS,3aR,8bR,8cR)-cannabicyclovarin (CBLV-C₃);

Cannabicitran-type (CBT): Cannabicitran (CBT-C₅); Cannabichromanone-type (CBCN): Cannabichromanone (CBCN—C₅), cannabichromanone-C3 (CBCN—C₃), cannabicoumaronone (CBCON—C₅).

In addition to the cannabinoids mentioned above, the crude drug also contains the associated carboxylic acids of said cannabinoids. These carboxylic acids are biosynthetic precursors.

Cannabis preparations have a variety of therapeutic effects, including antispastic, analgesic, antiemetic, neuroprotective, anti-inflammatory effects as well as effects for psychiatric disorders (Grotenhermen F, Müller-Vahl K: The therapeutic potential of cannabis and cannabinoids. Dtsch Arztebl Int 2012; 109(29-30): 495-501. DOI: 10.3238/arztebl.2012.0495).

Since 2011, a cannabis extract containing THC (dronabinol) and CBD in a 1:1 ratio (Nabiximols) has been legally approved in Germany for the treatment of moderate to severe, treatment-resistant spasticity in patients with multiple sclerosis (MS) as a sublingual spray (Sativex).

The chemical structures of some cannabinoid active substances and the nomenclature of the two active substances of the tetrahydrocannabinol, the IUPAC names of which are (6aR-trans)-6a,7,8,10a-tetrahydro-6,6,9-trimethyl-3-pentyl-6H-dibenzo[b,d]pyran-1-ol or Δ9-THC and (6aR-trans)-6a,7,10,10a-tetrahydro-6,6,9-trimethyl-3-pentyl-6H-dibenzo[b,d]pyran-1-ol or A8-THC are provided in the following.

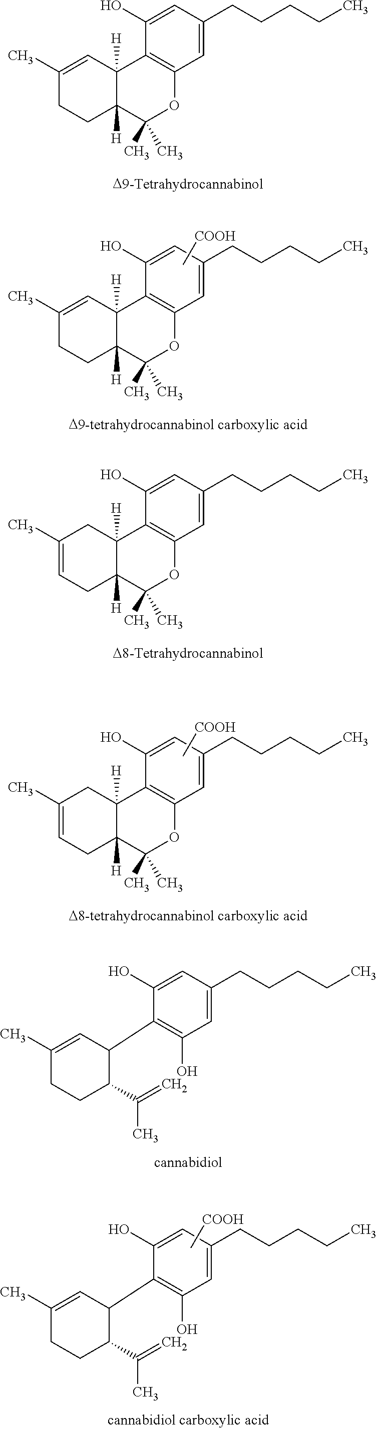

For the purposes of the present invention, unless otherwise specified, the term “tetrahydrocannabinol” or “THC” is intended to encompass all isomers, in particular double-bond isomers. THC can be produced synthetically (EP2314580B1).

DE 100 51 427 CI (Müller) describes a THC-containing primary extract obtained by CO₂ extraction from cannabis/hemp using super- or subcritical pressure and temperature conditions. To do this, a SFC or SFE system is used (supercritical fluid-chromatography).

A suitable short-path distillation in vacuum for extracting THC in high purity from a cannabis raw material is, however, not described in the state of the art. The simple vacuum distillation of THC is disclosed in the state of the art (WO 00/25127A1), and EP1051084B1 describes a steam distillation out of hemp.

WO2017055619A1 describes a short-path distillation for cannabidiol.

The object is therefore to provide an improved distillation for extracting THC, so that preferably a THC-enriched extract, even THC in high purity, is obtained.

The object is achieved with the claims.

Therefore, the invention relates to a method for extracting THC from cannabis plant material, wherein a short-path distillation is carried out in a vacuum (in the following: the method according to the invention).

In the context of this invention, distillation is a thermal separation process to obtain evaporable liquids from a gas phase and separate them from poorly evaporable substances, as a result of which a THC-containing extract is enriched in the distillate. “Vacuum distillation” in the context of this invention means that the distillation is carried out in a vacuum at 0.001 to 50 mbar, preferably 0.001 to 10 mbar, particularly preferably 0.001 to 1 mbar in the so-called fine vacuum range.

It is further preferred for the evaporator temperature to be 120 to 240 degrees Celsius, in particular 150 to 230 degrees Celsius.

It is essential that the method for vacuum distillation according to the invention takes place by means of a short-path distillation in a vacuum. “Short-path distillation in a vacuum” in the context of this invention means that the gas phase in the applied fine vacuum has to traverse only a very short path of preferably 10 cm, in particular 5 cm, in particular 2 cm, in particular 0.5 cm, between the evaporator wall and the (internal) condenser. A short-path evaporator can be used, for example, which corresponds structurally to a conventional thin film evaporator, but in which the condenser is integrated into the interior of the evaporator cylinder, so that the path that the vapors have to traverse to the condenser is very short and pressures of 0.001 mbar can be achieved.

A suitable thin film evaporator in the context of this invention comprises a substantially cylindrical, steam-heated inner wall, to which a thin film of a primary extract is applied by means of rotating distribution elements. The motor-driven distribution elements are needed to apply and distribute the mixture that evaporates rapidly on the plates.

In a further embodiment, a wiper system accelerates the evaporation process by keeping the thin film (product film) in a state of turbulence and optimizing the heat transfer and the mass transfer. The lower boiling fractions of the fed-in raw material or raw extract evaporate directly from the product film within a short period of time. The required dwell time of the product on the heated interior wall of the evaporator is therefore minimal. Thanks to the short dwell time, the low evaporation temperature and the immediate subsequent cooling of the concentrate, the thermal damage and loading of the cannabinoids, in particular THC, is minimized. It is particularly advantageous that the added raw extract can be applied without an additional solvent.

Preferred according to the invention, however, is a short-path evaporator or a correspondingly adapted thin film evaporator in a vacuum apparatus.

It is further preferred that the method for short-path distillation in a vacuum according to the invention is carried out with at least one additional column or separating column. A suitable column according to the invention is, for example, a DN 60 column (Ø50 mm, L=360 cm) with a vacuum jacket and TI nozzles made of borosilicate or stainless steel. The column can have conventional trays and also packing bodies or compartments, in order to preferably achieve at least 10 separation stages. Such separating columns according to the invention can be obtained from VTA Verfahrenstechnische Anlagen GmbH & Co. KG, Niederwinkling (DE) or UIC GmbH, Alzenau-Hörstein, for example. In a further embodiment, the length of the column is at least 2.50 m, preferably 2.70 m or more than 3 m.

In a further preferred embodiment of the invention, the short-path distillation in a vacuum can be provided with an additional column distillation. In particular, a short-path evaporator, or an appropriately adapted thin film evaporator, can be accordingly fitted with a separating column. In the context of this invention, a column distillation is likewise a suitable rectification in a vacuum, even with reflux, that preferably comprises 10 separation stages. The rectification permits the reliable separation of CBD and the enrichment of THC to more than 80%.

It is further preferred that the short-path distillation in a vacuum according to the invention is carried out using a coupled or connected column distillation or a coupled or connected separating column at a pressure of 0.001 to 50 mbar, preferably 0.001 to 10 mbar, particularly 0.001 to 1 mbar. It is also preferred for the temperature to be 120 to 240 degrees Celsius, in particular 150 to 230 degrees Celsius. In a preferred embodiment, the pressure is up to 5-10 mbar and the temperature is 200 to 230 degrees Celsius. This permits the reliable enrichment of THC, while other cannabinoids are completely depleted.

It is furthermore particularly preferred that the short-path distillation in a vacuum according to the invention is combined with a centrifugal partition chromatography (CPC) process, in series upstream or downstream. The short-path distillation in a vacuum can in particular be coupled or connected with a CPC process.

Therefore, in a further object, the invention relates to a method for separating and/or purifying natural substances from plant extracts, in particular cannabinoids from a cannabis extract.

To achieve this object, a method for separating and/or purifying natural substances from plant extracts, in particular cannabinoids from a cannabis extract, is carried out, which comprises at least one liquid-liquid partition chromatography step, wherein a solvent is held stationary by centrifugal force with a second immiscible liquid phase in the mobile phase and a short-path distillation in a vacuum takes place before or after, wherein one or more fractions or one or more pure substances are removed.

The CPC process can be used to obtain and enrich plant constituents from plant extracts in the analytical, semi-preparative and preparative scale. The CPC is a liquid-liquid chromatography method using a mostly two-phase solvent system.

The CPC process permits a virtually loss-free separation of highly complex substance mixtures from raw extracts.

Manufacturers of such centrifugal distribution chromatographs for carrying out a CPC are Kromaton S.a.r.l (Annonay, FR) or Armen Instrument Sas (Saint-Ave, FR), for example.

As with conventional liquid-liquid chromatography methods, such as high speed countercurrent chromatography (HSCCC), for example, a 2-phase solvent mixture is used for the CPC process. The upper or the lower phase can selectively be used as the stationary phase. Unlike HSCCC, however, CPC does not work with a capillary coil, but rather with a rotor provided with multiple hundred separation chambers. The distribution of the substances contained in the plant extract between the mobile phase and the stationary phase takes place in these chambers, which are connected directly one behind the other.

During separation, the system is set in fast rotation (up to 2,500 rpm). As a result, depending on the flow direction, on the one hand the desired phase is retained in the rotor of the CPC and, on the other hand, the separation of the two phases is accelerated by the centrifugal force. This permits the use of high flow rates and consequently the throughput of large amounts of substance in a short amount of time, so that a preparative application of this separation technique is possible.

It is particularly preferred that the short-path distillation in a vacuum according to the invention is combined with a sequential centrifugal partition chromatography (sCPC) process, in series upstream or downstream. The short-path distillation in a vacuum can in particular be coupled or connected with an sCPC process.

To achieve said abovementioned object, a method for separating and/or purifying natural substances from plant extracts, in particular cannabinoids from a cannabis extract, is carried out, which comprises at least one liquid-liquid partition chromatography step, wherein a continuous change of the stationary phase to the mobile phase and vice versa takes place and a short-path distillation in a vacuum takes place before or after, wherein one or more fractions or one or more pure substances are removed.

A characteristic feature of sCPC is the continuous change of the stationary phase to the mobile phase and vice versa, i.e. the denser or less dense phase can be selected to be the mobile phase and a change of this selection is possible during separation. Due to a different distribution coefficient, the substances to be separated are moved in a liquid-liquid centrifugal column at different velocities in the two phases having different densities.

An sCPC apparatus comprises at least one rotor having many round metal plates in which, for example, more than one thousand series-connected separation chambers are located (also referred to as rotor chambers, when forming a rotating separating column). With the aid of a pump, a mobile liquid phase (continuously) flows through the stationary liquid phase in the rotor. The rotor is accelerated to approx. 1,000 rpm and more, for example, so that, as a result of the density differences in the individual chambers, the centrifugal force causes the separation of the two liquid phases. As soon as the liquid mobile phase is in equilibrium with the liquid stationary phase, the sample or the substance mixture can be injected into the rotor.

The substance separation takes place as a result of differing adsorption in the mobile or stationary phase or the respective distribution coefficient (K) of a substance to the mobile/stationary phase, wherein the substances are moved through the individual separation chambers to the rotor outlet and fractionated. The characteristic alternating change of the stationary phase and the mobile phase takes place by controlling the used pumps with the aid of valves in a frequency to be selected. In a period of time to be selected (duration of pumping, pump duration), the associated solvent is pumped into the respectively driven phase, namely in each case at the two ends of a rotor, so that the pumps are oppositely disposed.

The sample or the mixture of substances is preferably fed into the middle of the rotor. In a further embodiment, two or more rotors can be coupled.

The rotor chambers are preferably filled with an upper and a lower liquid phase in a 50/50 ratio (volume %). The substance mixture is continuously fed between the two rotating separating columns with the aid of a pump at a defined flow rate and the separation takes place in a cyclical and time-delayed manner, whereby, in a first step, the upper phase serves as the mobile phase (ascending) and, in a second step, the lower phase serves as the mobile phase (descending).

In accordance with the concentration and the different distribution coefficients of the substances in the substance mixture, the separation takes place in two product streams.

Suitable devices and equipment can be obtained from Armen Technologies (France) under the name “True Moving Bed CPC”.

The sCPC process can be used to obtain and enrich plant constituents from plant extracts in the analytical, semi-preparative and preparative scale. The sCPC is a liquid-liquid chromatography method using a mostly two-phase solvent system.

According to the invention, the following genera are preferred for plant extracts:

Equiseti, Juglandis, Millefolii, Quercus, Taraxaci, Althaeae, Matricariae, Centaurium, Levisticum, Rosmarinus, Angelica, Artemisia, Astragalus, Leonurus, Salvia, Saposhnikovia, Scutellaria, Siegesbeckia, Armoracia, Capsicum, Cistus, Echinacea, Galphimia, Hedera, Melia, Olea, Pelargonium, Phytolacca, Primula, Salix, Thymus, Vitex, Vitis, Rumicis, Verbena, Sambucus, Gentiana, Cannabis, Silybum.

According to the invention, the following species are preferred for plant extracts:

Equiseti herba (horsetail), Juglandis folium (walnut leaf), Millefolii herba (yarrow), Quercus cortex (oak bark), Taraxaci herba (dandelion), Althaeae radix (marshmallow root) and Matricariae flos (or Flos chamomillae (chamomile)) Centaurium erythraea (centaury), Levisticum officinale (lovage), Rosmarinus officinalis (rosemary), Angelica dahurica (Dahurian angelica, Pinyin name: Bai Zhi), Angelica sinensis (Chinese angelika, Pinyin name: Dang Gui), Artemisia scoparia (capillary wormwood, Pinyin name: Yin Chen), Astragalus membranaceus (var. Mongolicus) (Mongolian milkvetch, Chin.: Huang-Qi), Leonurus japonicus (Oriental motherwort, Chin.: T'uei), Salvia miltiorrhiza (red sagei, Chin.: Danshen), Saposhnikovia divaricata (siler, Pinyin name: Fang Feng), Scutellaria baicalensis (Baikal skullcap), Siegesbeckia pubescens (Pinyin name: Xi Xian Cao), Armoracia rusticana (horseradish), Capsicum sp. (pepper), Cistus incanus (hoary rock-rose), Echinacea angustifolia (narrow-leaved purple coneflower), Echinacea purpurea (purple coneflower), Galphimia glauca, Hedera helix (ivy), Melia toosendan (Chinese elderberries, Chin.: Chuan Lian Zi), Olea europaea (olive), Pelargonium sp. (pelargonia), Phytolacca americana (pokeweed), Primula veris (cowslip), Salix sp. (willow), Thymus L. (thyme) Vitex agnus castus (chasteberry), Vitis vinifera (common grape vine), Rumicis herba (sorrel herb), Verbena officinalis (verbena), Sambucus nigra (black elder), Gentiana lutea (yellow gentian), Cannabis sativa (hemp), Silybum marianum (milk thistle).

Mixtures of the aforementioned genera and/or species are likewise included in the invention.

The abovementioned species and genera are particularly rich in healing natural substances and are described as medicinal plants, for example as in the plant extract-based products of Bionorica SE (e.g. Bronchipret®, Imupret®, Sinupret®). Such plants also contain common characteristic substance classes such as flavonoids, polyphenols, etc.

In a preferred embodiment, the plant extract is obtained from a first solvent such as alcohols, ethanol, water, hydrocarbons, heptane or mixtures thereof, and the soluble components are used.

For example, in the aforementioned methods, these substance classes of plant extracts, such as alkaloids, bitter compounds, anthocyanins, anthraquinones, coumarins, flavonoids, glucosinolates, lactones, lignans, lipids, cannabinoids, phenols, polyphenols, saponins, terpenes, xanthones, can be enriched or depleted in the fractions, which can be removed.

In the aforementioned methods, such fractions and pure substances can particularly advantageously be obtained in high purity and yield.

Solvents that can be used in the liquid-liquid partition chromatography can, for example, be found in Skalicka-Wozniak K, Garrard I, A comprehensive classification of solvent systems used for natural product purifications in countercurrent and centrifugal partition chromatography, Nat Prod Rep. 2015 November; 32(11):1556-61.

According to the invention, examples of suitable solvents from which two-phase solvent systems (mobile phase/stationary phase) can be provided for plant extracts are:
a.) hydrocarbons such as n-hexane, cyclohexane, isohexane, heptane, isooctane;
b.) ethers such as t-butyl methyl ether, petroleum ether, diethyl ether;
c.) halogenated solvents such as chloroform, dichloromethane, benzotrifluoride, dichloroethane, tetrachloromethane, trichlorethane;
d.) water soluble alcohols such as butanol, methanol, ethanol isopropanol;
e.) water soluble esters such as ethyl acetate, isopropyl acetate;
f.) acetonitrile, toluene.

Suitable two-phase solvent systems can be produced from the aforementioned solvents, preferably such as:

n-heptane/acetonitrile

n-heptane/ethyl acetate/acetonitrile;

n-heptane/ethyl acetate/t-butyl methyl ether/acetonitrile; n-heptane/ethyl acetate/methanol/water;

n-heptane/ethanol/water.

Therefore, according to the invention, a method for separating and/or purifying natural substances from plant extracts, in particular cannabinoids from a cannabis extract, in particular THC from cannabis plant material, is provided, wherein a short-path distillation in a vacuum is coupled with or preceded or followed by a CPC process and/or sCPC.

For example, for the extraction of THC, the CPC process comprises at least one liquid-liquid partition chromatography step or the use of a centrifugal distribution chromatograph for liquid-liquid partition chromatography, whereby a solvent, which is held stationary by centrifugal force, and through which a second immiscible liquid phase can be pumped as the mobile phase, is preferably selected from hexane, cyclohexane, heptane, n-heptane, iso-heptane, octane, n-octane, iso-octane.

For example, for the extraction of THC, the sCPC process comprises at least one liquid-liquid partition chromatography step, whereby a solvent is preferably selected from hexane, cyclohexane, heptane, n-heptane, iso-heptane, octane, n-octane, iso-octane, and a continuous change of the stationary phase to the mobile phase and vice versa takes place.

Both the density and the viscosity of hexane, cyclohexane, n-heptane and iso-octane are greater than the density/viscosity of n-hexane, such that, for example with respect to the second mobile phase, e.g. acetonitrile, a more stable two-phase system is produced, so that a better retention of the stationary phase is made possible and consequently an increased separation efficiency is achieved; for example, with a purity for THC of more than 95 wt %, even 99 wt %.

In a further embodiment, one or more short-path distillations in a vacuum can be connected upstream or downstream, so that, for example, a purity for THC of more than 99.0 wt %, in particular more than 99.7 wt %, can be achieved.

It is furthermore preferred that the short-path distillation in a vacuum takes place on a first primary extract having at least 15 wt % of THC or a desired cannabinoid. In a first step, therefore, the cannabis plant material is cut, comminuted and subjected to a first extraction, for example a CO2 extraction, as described in DE 100 51 427 CI. An extraction can alternatively also be carried out with an alkane, propane, butane, pentane, hexane, heptane, alcohol, methanol, ethanol, propanol, isopropanol, water, and other liquid and gaseous solvents or a column chromatography, as a result of which a primary extract can be obtained, which preferably has at least 15 wt % THC and is used for further short-path distillation in a vacuum (supra). A CO₂-extraction is preferred, however. Drug hemp (Cannabis sativa) is the preferred cannabis plant material.

The carboxylic acids of the cannabinoids, in particular tetrahydrocannabinol carboxylic acids, cannabidiol carboxylic acid, can be decarboxylated in the primary extract as well. The decarboxylation is carried out by heating at 120° C. under a vacuum, preferably at 100-200 mbar. A continuous decarboxylation using short-path distillation in the degassing stage is likewise possible at preferably 120° C. and 1 mbar.

A THC-containing extract that can be obtained using a method according to the invention is therefore also a subject matter of the invention.

In a further embodiment according to the method according to the invention, it was possible to obtain an extract having the following composition after a short-path distillation in a vacuum:

| | | |
|---|---|---|
| Delta9-THC | WT % | 92.04 |
| CBC | WT % | 0.83 |
| CBG | WT % | 1.51 |
| CBN | WT % | 0.73 |
| VUX | WT % | 0.00 |

The method according to the invention therefore permits an enrichment of THC of more than 90 weight %, in particular 92 weight %, as well as also 95 weight % or more, along with an advantageous depletion of other cannabinoids and difficult-to-remove impurities.

Furthermore, with respect to THC [wt %], the low boiling fraction is 5 and, with respect to THC [wt %], the high boiling fraction is 3. In other words, using the method according to the invention, it was advantageously possible to remove almost all the substances that boil before and after THC.

Therefore, the invention also relates to a THC-containing extract, which can be obtained using a method according to the invention and comprises a high boiling fraction of less than 53 wt % with respect to THC and a low boiling fraction of less than 5 wt % with respect to THC.

An exemplary starting composition was furthermore used for the method according to the invention as follows:

| | | |
|---|---|---|
| Delta 9-THC | WT % | 78 |
| CBC | WT % | 0.69 |
| CBG | WT % | 1.36 |
| CBN | WT % | 0.62 |

The method according to the invention particularly advantageously permits the significant reduction of the low and high boiling fractions in relation to a used starting material. This results in an extract with specific properties, such as advantageous residue-free evaporability, which is particularly suitable for the inhalation of THC in medical applications, in particular for a medicinal product. The residue-free evaporability of THC is in particular obtained at 001 to 1 mbar. It is further preferred for the temperature to be 120 to 240 degrees Celsius, in particular 150 to 230 degrees Celsius, in particular 180-200 degrees Celsius.

Said extract can be particularly advantageously used for CPC or sCPC because, compared to a non-distilled extract, the loading can be increased by factor of 5.

The obtained fractions, pure substances, extracts can be the subject matter of a galenic formulation or a pharmaceutical composition.

The galenic preparation of a pharmaceutical composition or agent according to the invention, particularly a medicinal product (medication), can be carried out in any manner that is customary in the state of the art. Suitable solid or liquid galenic preparation forms include granulates, powders, dragées, tablets, (micro) capsules, hard capsules, suppositories, syrups, juices, suspensions or emulsions, for the preparation of which common additives, such as carrier substances, disintegrants, binders, coatings, swelling agents, glidants or lubricants, flavorings, sweeteners and solubilizers are used. Excipients include magnesium stearate, sodium chloride, magnesium carbonate, titanium dioxide, lactose, mannitol and other sugars, talcum, milk protein, gelatins, starch, cellulose and its derivatives, animal and vegetable oils, such as cod liver oil, sunflower, peanut or sesame oil, polyethylene glycols, and solvents, such as sterile water and monohydric or polyhydric alcohols, for example glycerol.

Other detergents and surfactants, for example as described below for a cosmetic composition, can be provided as excipients and additives as well.

Emulsions are generally understood to be heterogeneous systems, which consist of two immiscible or only limitedly miscible liquids that are typically referred to as phases. In an emulsion, one of the two liquids is dispersed in the other liquid in the form of very fine droplets. If the two liquids are water and oil and oil droplets are finely distributed in water, it is an oil-in-water emulsion (O/W emulsion). The basic characteristics of an O/W emulsion are influenced by the water. A water-in-oil emulsion (W/O emulsion) is the same principle in reverse, in which the basic characteristics are determined by the oil. Mixed systems, such as water-in-oil-in-water emulsions (W/O/W emulsion) and oil-in-water-in-oil emulsions (O/W/O emulsions), are known as well. All the mentioned emulsions are suitable according to the invention.

The water-free systems according to the invention include pure oil preparations, such as skin oils. Pastes that can also be used contain the preparation according to the invention and are characterized by the fact that they consist of the same or similar components as an emulsion, but are substantially free of water. Within the context of the present invention, the terms oil phase and lipid phase are used synonymously. In another preferred embodiment, the preparation according to the invention can contain an emulsifier as a further component. In a very preferred embodiment, this emulsifier can be an O/W emulsifier.

Emulsifiers can advantageously be selected from the group of nonionic, anionic, cationic or amphoteric emulsifiers.

Various emulsifiers from the groups of the partial fatty acid esters, fatty alcohols, sterols, polyethylene glycols such as ethoxylated fatty acids, ethoxylated fatty alcohols, and ethoxylated sorbitan esters, sugar emulsifiers, polyglycerol emulsifiers or silicone emulsifiers can be used as the nonionic emulsifier.

Various emulsifiers from the groups of the soaps, e.g. sodium stearate, fatty alcohol sulfates, mono-, di- and trialkylphosphoric acid ester and their ethoxylates, fatty acid lactate ester, fatty acid citrate ester, or fatty acid citroglycerol ester, can be used as the anionic emulsifier.

Quaternary ammonium compounds having a long-chain aliphatic radical, e.g. distearyldimonium chlorides, can be used as the cationic emulsifiers.

Various emulsifiers from the groups alkylamininoalkane carboxylic acids, betaines, sulfobetaines or imidazoline derivatives can be used as amphoteric emulsifiers.

Preferred according to the invention are naturally occurring emulsifiers, including beeswax, lanolin wax, lecithin and sterols, for example among others, which can likewise be used in the production of a preparation according to the invention In a preferred formulation of the preparation according to the invention, O/W emulsifiers can be selected from the group of plant protein hydrolysates and their derivatives.

In the context of the present invention, substances selected from the group of esters of saturated and/or unsaturated, branched and/or unbranched alkane carboxylic acids and/or alkene carboxylic acids having a chain length of 3-30 carbon atoms and saturated and/or unsaturated, branched and/or unbranched alcohols having a chain length of 3-30 carbon atoms and from the group of esters of aromatic carboxylic acids and saturated and/or unsaturated, branched and/or unbranched alcohols having a chain length of 3 to 30 carbon atoms can also advantageously be contained as additives. Esterols of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, such as jojoba oil.

The oil phase can further advantageously be selected from the group of branched and unbranched hydrocarbons and waxes, dialkyl ethers, the group of saturated or unsaturated, branched or unbranched alcohols, and fatty acid triglycerides, particularly triglycerol esters of saturated and/or unsaturated, branched and/or unbranched alkane carboxylic acids having a chain length of 8-24 C atoms, in particular 12-18 C atoms. The fatty acid triglycerides can, for example, advantageously be selected from the group of synthetic, semisynthetic and natural oils.

Antioxidants and/or radical catchers can in particular additionally be added to the preparations according to the invention as an excipient or an additive. Such antioxidants are advantageously selected from the group of lipophilic systems, for example: natural and synthetic tocopherols, nordihydroguaiaretic acid, coniferyl benzoate, butylhydroxyanisole, butylhydroxytoluene, gallic acid ester, and various antioxidative plant extracts. Among the hydrophilic systems, it is particularly advantageous to use inorganic sulfur compounds, sodium hydrogen sulfite, cysteine or ascorbic acid.

The following examples serve to further illustrate the invention, however without limiting the invention to said examples.

### EXAMPLE 1

### 1.1 Short-Path Distillation V9001

Cannabis extract 50-95% Cannabis sativa was used as the educt. The educt was previously heated at 80° C. in the oil bath and transferred to the heated drip funnel of the short-path distillation. The used quantity was 103.3 g. The temperature in the dosing vessel was 70° C. The speed of the stirrer was 400 rpm and the temperature in the evaporator was 180° C. The extract was fed in with a speed of 180 ml/h-200 ml/h and the pressure in the overall apparatus was 1.2*10-1 mbar. The distillation took a total of 35 minutes. It was possible to obtain 79.53 g distillate and 58.15 g residue, which corresponds to a cut ratio of approximately 75/25. During distillation, a distillate additionally formed on the cold trap. The quantity of distillate was approximately 3 ml. A hot air dryer was needed to get the resulting residue into a flowable form. The distillate, which was subsequently used further, had a bright yellow color.

### 1.2 Short-Path Distillation V9002

137.51 g cannabis extract 50-95% Batch 0000106433 was used. The temperature in the dosing vessel was 70° C., the temperature in the evaporator was 165° C. As before, the speed was set to 400 rpm. The entire distillation took about 45 minutes. It was possible to obtain 90.4 g distillate, while 61.31 g of residue formed. Approximately 3 ml of a distillate formed in the cold trap. The cut ratio was 65/35. The distillate, which was subsequently used further, had a bright yellow color.

### 1.3 Preparation of the Stock Solution V9001

The distillate from the short-path distillation was used as the starting material for the stock solution. The same concentration was used for both the stock solution and the FCPC system, i.e. 0.1 g/ml. The distillate from V9001 was dissolved in the water bath at 80° C., then transferred into a 1000 ml round-bottom flask. The weight was 62.26 g. The 1000 ml round-bottom flask was then again dissolved in a water bath at 80° C. and mixed with 622 ml heptane FCPC and dissolved.

The results are shown in Table 1.

### EXAMPLE 2

Comparison test of a THC-containing cannabis extract, with and without short-path vacuum distillation (short path: 2-5 cm)

| Information provided | | |
|---|---|---|
| in wt % | without SPD | with SPD |
| D9-THC | 97.53 | 98.90 |
| Impurity A | 0.00 | 0.05 |
| Impurity B | 0.12 | 0.00 |
| Impurity C | 0.09 | 0.08 |
| Impurity D | 0.32 | 0.24 |
| Impurity E | 0.05 | 0.06 |
| CBD | 0.00 | 0.00 |
| CBN | 0.05 | 0.10 |
| CBC | 0.64 | 0.62 |
| Exo-THC | <0.05 | <0.05 |
| D8-THC | 0.00 | 0.00 |
| Other impurities | 1.30 | <0.05 |

Impurities can be significantly minimized.

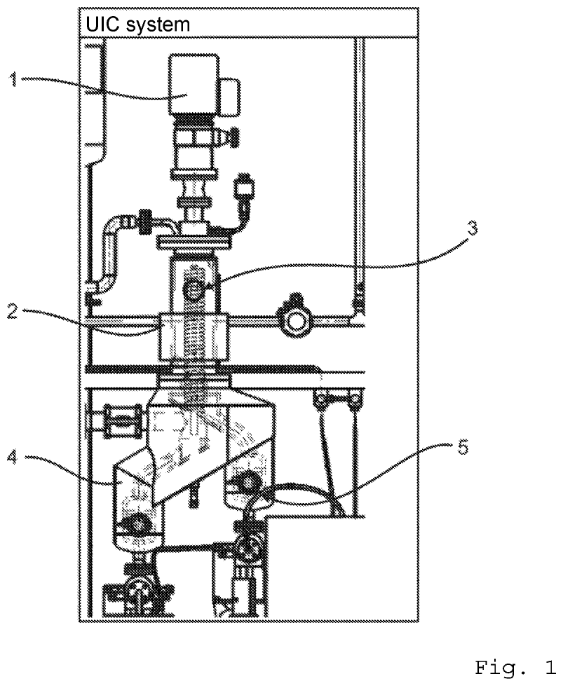

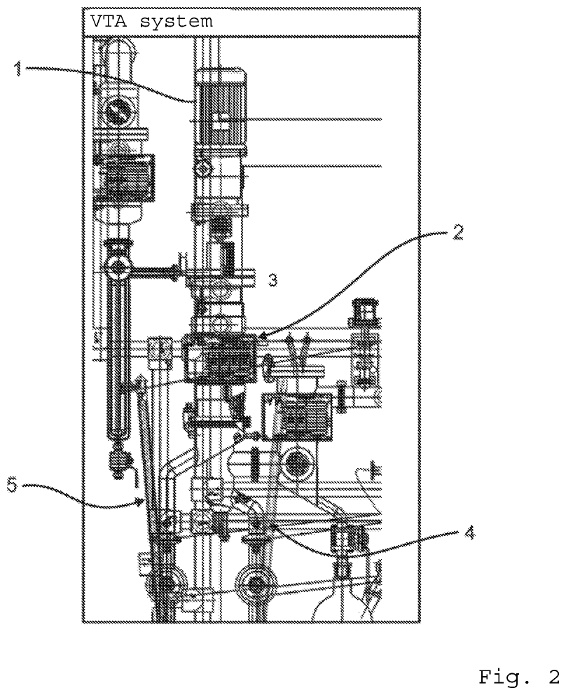

FIGS. 1 and 2 show a cross section of suitable systems for short-path vacuum distillation with the following reference signs:
1 Wiper motor
2 Evaporator
3 (Internal) condenser
4 Distillate discharge
5 Residue discharge

TABLE 1

| | | | Test No. Info | | | | | | | | | | |
|---|---|---|---|---|---|---|---|---|---|---|---|---|---|
| | | | V9001 | V9001 | V9001 | V9002 | V9002 | V9002 | V9005 | V9005 | V9005 | V9006 | V9006 |
| Description | NTHC KDL 1 | CW46, 2016 | Start | — | End | Start | — | End | Start | — | End | Start | — |
| Feed, from protocol | Cannabis extract | | | | | | | | | | | | |
| | 50-95% | | | | | | | | | | | | |
| Batch B: | 0000106433 | | | | | | | | | | | | |
| Date | | mm/dd/yyyy | 11/16/2016 | 11/16/2016 | 11/16/2016 | 11/17/2016 | | 11/17/2017 | 01/13/2017 | | 01/13/2017 | 01/13/2017 | |
| Time | | hh:mm | 1:50 p.m. | 2:00 pm | 2:45 pm | 9:43 a.m. | | 10:30 a.m. | 10:47 a.m. | | 11:30 a.m. | 11:30 a.m. | |
| Dosing vessel | | ° C. | 70 | 70 | 70 | 70 | | 70 | 70 | | 70 | 70 | |
| Evaporator | | ° C. | 180 | 180 | 180 | 165 | | 165 | 195 | | 195 | 215 | |
| Vacuum | | mbar | 0.17-0.18 | 0.12 | 0.12 | 0.12 | | 0.12 | 0.17-0.18 | | 0.17-0.18 | | |
| Speed | | RPM | 400 | 400 | 400 | 400 | | 400 | 400 | | 400 | 400 | |
| Feed, set | | ml/h | 180-200 | 180-200 | 200 | 180-200 | | 180-200 | 180 | | 180 | — | |
| Feed, protocol | | g/h | — | — | — | — | | — | — | | — | — | |
| Distillation time | | min | | | 35 | | | 47 | | | 43 | | |
| Feed calculated | | g/h | | | 177 | | | 176 | | | 136 | | |
| m (educt, start) | | g | | | 800.26 | | | 692.85 | | | 527.00 | | |
| m (educt, after removal) | | g | | | 696.96 | | | 555.34 | | | 429.27 | | |
| m (educt, consumed), feed F | | g | | | 103.30 | | | 137.51 | 215.529 | | 97.73 | | |
| m (distillate) + m (flask) | | g | | | 127.39 | | | 138.56 | | | 126.93 | | |
| m (distillate flask) | tare | g | | | 47.86 | | | 48.14 | | | 49.28 | | |
| m (distillate) | Distillate D | g | | | 79.53 | | | 90.42 | | | 77.65 | | |
| m (residue) + m (flask) | | g | | | 58.15 | | | 61.31 | | | 54.02 | | |
| m (residue flask) | tare | g | | | 47.77 | | | 48.11 | | | 47.77 | | |
| m (residue) | Residue R | g | | | 23.77 | | | 47.09 | | | 6.25 | | |
| m (cold trap) | Cold trap K | g | | | | | | | | | | | |
| m (cold trap/feed) | K/F | % | | | | | | | | | | | |
| Cut ratio D/R | D/R It. Protocol | % | | | 75/25 | | | 65/35 | | | | | |
| Cut ratio | D/F calculated | % | | | 77 | | | 66 | | | 79 | | |
| | | | Test | | | | | | | | | | |
| | Mass fraction | | V9001 | V9001 | V9001 | V9002 | V9002 | V9002 | V9005 | V9005 | V9005 | V9006 | V9006 |
| Analysis | [%] | Current | F | D | R | F | D | R | F | D | R | F | D |
| CBG | | % | 1.36 | 1.67 | 0.52 | 1.36 | 1.51 | 0.67 | 1.36 | | | 1.36 | |
| CBN | | % | 0.62 | 0.81 | 0.21 | 0.62 | 0.73 | 0.27 | 0.62 | | | 0.62 | |
| D9-THC | | % | 78.84 | 88.73 | 16.54 | 78.84 | 92.04 | 18.78 | 78.84 | 85.00 | 6.70 | 78.84 | 81.40 |
| D8-THC | | % | 0.08 | 0.13 | 0.04 | 0.08 | 0.02 | 0.04 | 0.08 | | | 0.08 | |
| CBC | | % | 0.69 | 0.85 | 0.11 | 0.69 | 0.83 | 0.14 | 0.69 | | | 0.69 | |
| VU74 at RT 74 | | % | 0.00 | 0.00 | 0.46 | 0.00 | 0.00 | 0.91 | 0.00 | | | 0.00 | |
| VU77 at RT 77 | | % | 0.00 | 0.00 | 0.53 | 0.00 | 0.00 | 1.09 | 0.00 | | | 0.00 | |
| Vux at RT −150 | | % | 0.20 | 0.00 | 0.20 | 0.20 | 0.00 | 1.33 | 0.20 | | | 0.20 | |
| | | | Test | | | | | | | | | | |
| | Absolute mass | | V9001 | V9001 | V9001 | V9002 | V9002 | V9002 | V9005 | V9005 | V9005 | V9006 | V9006 |
| Evaluation | [g] | Current | F | D | R | F | D | R | F | D | R | F | D |
| CBG | | g | 1.40 | 1.32 | 0.12 | 1.87 | 1.37 | 0.32 | 1.33 | 0.00 | 0.00 | 1.60 | 0.00 |
| CBN | | g | 0.64 | 0.65 | 0.05 | 0.85 | 0.66 | 0.13 | 0.60 | 0.00 | 0.00 | 0.73 | 0.00 |
| D9-THC | | g | 81.44 | 70.57 | 3.93 | 108.41 | 83.23 | 8.85 | 77.04 | 66.00 | 0.42 | 92.87 | 81.05 |
| D8-THC | | g | 0.09 | 0.10 | 0.01 | 0.11 | 0.02 | 0.02 | 0.08 | 0.00 | 0.00 | 0.10 | 0.00 |
| CBC | | g | 0.72 | 0.68 | 0.03 | 0.96 | 0.75 | 0.07 | 0.68 | 0.00 | 0.00 | 0.82 | 0.00 |
| VU74 at RT 74 | | g | 0.00 | 0.00 | 0.11 | 0.00 | 0.00 | 0.43 | 0.00 | 0.00 | 0.00 | 0.00 | 0.00 |
| VU77 at RT 77 | | g | 0.00 | 0.00 | 0.13 | 0.00 | 0.00 | 0.52 | 0.00 | 0.00 | 0.00 | 0.00 | 0.00 |
| Vux at RT −150 | | g | 0.21 | 0.00 | 0.05 | 0.28 | 0.00 | 0.63 | 0.20 | 0.00 | 0.00 | 0.24 | 0.00 |
| | | | Test | | | | | | | | | | |
| | Enrichment | | | V9001 | | | V9002 | | | V9005 | | | V9006 |
| Yield in distillate | [%] | Current | | D | | | D | | | D | | | D |
| CBG | VU | % | | 94.42 | | | 73.16 | | | 0.00 | | | 0.00 |
| CBN | VU | % | | 100.00 | | | 78.17 | | | 0.00 | | | 0.00 |
| D9-THC | Product | % | | 86.65 | | | 76.77 | | | 85.67 | | | 87.27 |
| D8-THC | VU | % | | 100.00 | | | 16.75 | | | 0.00 | | | 0.00 |
| CBC | VU | % | | 94.60 | | | 78.14 | | | 0.00 | | | 0.00 |
| VU74 at RT 74 | VU | % | | — | | | — | | | — | | | — |
| VU77 at RT 77 | VU | % | | — | | | — | | | — | | | — |
| Vux at RT −150 | VU | % | | 0.00 | | | 0.00 | | | 0.00 | | | 0.00 |
| Mass balance D9-THC | TARGET = 0 | g | | 6.9 | | | 16.3 | | | 10.6 | | | 11.6 |
| Mass balance D9-THC | Deviation in % | % | | 8.5 | | | 15.1 | | | 13.8 | | | 12.5 |
| Summary D9 THC in feed (educt) | | | | V9001 | | | V9002 | | | V9005 | | | V9006 |
| w (D9-THC) feed | w(D9-THC) | % | | 78.8 | | | 78.8 | | | 78.8 | | | 78.8 |
| Summary D9-THC in the product | | | | V9001 | | | V9002 | | | V9005 | | | V9006 |
| Cut ratio | D/F | % | | 77.0 | | | 65.8 | | | 79.5 | | | 84.5 |
| w (D9-THC) product | w(D9-THC) | % | | 88.7 | | | 92.0 | | | 85.0 | | | 81.4 |
| Yield D9-THC | m(D9,D)/m(D9,F) | % | | 86.7 | | | 76.8 | | | 85.7 | | | 87.3 |

% = wt %

1. A method for extracting tetrahydrocannabinol from cannabis plant material, characterized in that a short-path vacuum distillation is carried out.

2. The method for extracting tetrahydrocannabinol from cannabis plant material according to claim 1, characterized in that the short-path vacuum distillation is carried out using a short-path evaporator.

3. The method for extracting tetrahydrocannabinol from cannabis plant material according to claim 1, characterized in that the length of the short path is 10 cm, in particular 5 cm, in particular 2 cm, in particular 0.5 cm between the evaporator wall and the condenser.

4. The method for extracting tetrahydrocannabinol from cannabis plant material according to claim 1, characterized in that the short-path vacuum distillation is carried out using an additional column or a separating column.

5. The method for extracting tetrahydrocannabinol from cannabis plant material according to claim 4, characterized in that the length of the separating column is at least 2.50 m.

6. The method for extracting tetrahydrocannabinol from cannabis plant material according to claim 1, wherein the vacuum distillation is carried out on a first primary extract having at least 15 wt % tetrahydrocannabinol.

7. The method for extracting tetrahydrocannabinol from cannabis plant material according to claim 1, wherein the pressure is 0.001 to 50 mbar and the temperature is 120 to 240 degrees Celsius.

8. A method for extracting tetrahydrocannabinol from cannabis plant material, characterized in that one or more short-path distillations in a vacuum take place before or after at least one liquid-liquid partition chromatography step.

9. A method for extracting tetrahydrocannabinol from cannabis plant material according to claim 8, characterized in that one or more short-path distillations in a vacuum take place before or after at least one liquid-liquid partition chromatography step, wherein at least i.) a continuous change of the stationary phase to the mobile phase and vice versa takes place and/or ii.) a solvent is held stationary by centrifugal force with a second immiscible liquid phase in the mobile phase.

10. A Tetrahydrocannabinol-containing extract according to claim 1, characterized in that the extract has a residue-free evaporability.

11. A medicinal product, nutritional supplement containing a tetrahydrocannabinol-containing extract which can be obtained using the method according to claim 1, in particular for inhalation.

12. A method for separating and/or purifying natural substances from plant extracts, in particular cannabinoids from a cannabis extract, comprising at least one liquid-liquid partition chromatography step, wherein a solvent is held stationary by centrifugal force with a second immiscible liquid phase in the mobile phase and one or more short-path distillations in a vacuum take place before or after, wherein one or more fractions or one or more pure substances are removed.

13. A method for separating and/or purifying natural substances from plant extracts, in particular cannabinoids from a cannabis extract, comprising at least one liquid-liquid partition chromatography step, wherein a continuous change of the stationary phase to the mobile phase and vice versa takes place and one or more short-path distillations in a vacuum take place before or after, wherein one or more fractions or one or more pure substances are removed.

14. The method for separating and/or purifying natural substances from plant extracts according to claim 12, characterized in that the fraction(s) contain such substance classes of plant extracts selected from the group alkaloids, bitter compounds, anthocyanins, anthraquinones, coumarins, flavonoids, glucosinolates, lactones, lignans, lipids, cannabinoids, phenols, polyphenols, saponins, terpenes, xanthones.

15. The method for separating and/or purifying natural substances from plant extracts according to claim 12, characterized in that at least one plant extract is selected from the group including the genera Equiseti, Juglandis, Millefolii, Quercus, Taraxaci, Althaeae, Matricariae, Centaurium, Levisticum, Rosmarinus, Angelica, Artemisia, Astragalus, Leonurus, Salvia, Saposhnikovia, Scutellaria, Siegesbeckia, Armoracia, Capsicum, Cistus, Echinacea, Galphimia, Hedera, Melia, Olea, Pelargonium, Phytolacca, Primula, Salix, Thymus, Vitex, Vitis, Rumicis, Verbena, Sambucus, Gentiana, Cannabis, Silybum.

16. The method for separating and/or purifying natural substances from plant extracts according to claim 15, characterized in that at least one plant extract is selected from the group including the species Equiseti herba (horsetail), Juglandis folium (walnut leaf), Millefolii herba (yarrow), Quercus cortex (oak bark), Taraxaci herba (dandelion), Althaeae radix (marshmallow root) and Matricariae flos (or Flos chamomillae (chamomile)) Centaurium erythraea (centaury), Levisticum officinale (lovage), Rosmarinus officinalis (rosemary), Angelica dahurica (Dahurian angelica, Pinyin name: Bai Zhi), Angelica sinensis (Chinese angelika, Pinyin name: Dang Gui), Artemisia scoparia (capillary wormwood, Pinyin name: Yin Chen), Astragalus membranaceus (var. Mongolicus) (Mongolian milkvetch, Chin.: Huang-Qi), Leonurus japonicus (Oriental motherwort, Chin.: T'uei), Salvia miltiorrhiza (red sagei, Chin.: Danshen), Saposhnikovia divaricata (siler, Pinyin name: Fang Feng), Scutellaria baicalensis (Baikal skullcap), Siegesbeckia pubescens (Pinyin name: Xi Xian Cao), Armoracia rusticana (horseradish), Capsicum sp. (pepper), Cistus incanus (hoary rock-rose), Echinacea angustifolia (narrow-leaved purple coneflower), Echinacea purpurea (purple coneflower), Galphimia glauca, Hedera helix (ivy), Melia toosendan (Chinese elderberries, Chin.: Chuan Lian Zi), Olea europaea (olive), Pelargonium sp. (pelargonia), Phytolacca americana (pokeweed), Primula veris (cowslip), Salix sp. (willow), Thymus L. (thyme) Vitex agnus castus (chasteberry), Vitis vinifera (common grape vine), Rumicis herba (sorrel herb), Verbena officinalis (verbena), Sambucus nigra (black elder), Gentiana lutea (yellow gentian), Cannabis sativa (hemp), Silybum marianum (milk thistle).

17. The method for extracting tetrahydrocannabinol from cannabis plant material according to claim 2, characterized in that the length of the short path is 10 cm, in particular 5 cm, in particular 2 cm, in particular 0.5 cm between the evaporator wall and the condenser.

18. The method for separating and/or purifying natural substances from plant extracts according to claim 13, characterized in that the fraction(s) contain such substance classes of plant extracts selected from the group alkaloids, bitter compounds, anthocyanins, anthraquinones, coumarins, flavonoids, glucosinolates, lactones, lignans, lipids, cannabinoids, phenols, polyphenols, saponins, terpenes, xanthones.

19. The method for separating and/or purifying natural substances from plant extracts according to claim 13, characterized in that at least one plant extract is selected from the group including the genera Equiseti, Juglandis, Millefolii, Quercus, Taraxaci, Althaeae, Matricariae, Centaurium, Levisticum, Rosmarinus, Angelica, Artemisia, Astragalus, Leonurus, Salvia, Saposhnikovia, Scutellaria, Siegesbeckia, Armoracia, Capsicum, Cistus, Echinacea, Galphimia, Hedera, Melia, Olea, Pelargonium, Phytolacca, Primula, Salix, Thymus, Vitex, Vitis, Rumicis, Verbena, Sambucus, Gentiana, Cannabis, Silybum.

20. The method for separating and/or purifying natural substances from plant extracts according to claim 18, characterized in that at least one plant extract is selected from the group including the species Equiseti herba (horsetail), Juglandis folium (walnut leaf), Millefolii herba (yarrow), Quercus cortex (oak bark), Taraxaci herba (dandelion), Althaeae radix (marshmallow root) and Matricariae flos (or Flos chamomillae (chamomile)) Centaurium erythraea (centaury), Levisticum officinale (lovage), Rosmarinus officinalis (rosemary), Angelica dahurica (Dahurian angelica, Pinyin name: Bai Zhi), Angelica sinensis (Chinese angelika, Pinyin name: Dang Gui), Artemisia scoparia (capillary wormwood, Pinyin name: Yin Chen), Astragalus membranaceus (var. Mongolicus) (Mongolian milkvetch, Chin.: Huang-Qi), Leonurus japonicus (Oriental motherwort, Chin.: Tuei), Salvia miltiorrhiza (red sagei, Chin.: Danshen), Saposhnikovia divaricata (siler, Pinyin name: Fang Feng), Scutellaria baicalensis (Baikal skullcap), Siegesbeckia pubescens (Pinyin name: Xi Xian Cao), Armoracia rusticana (horseradish), Capsicum sp. (pepper), Cistus incanus (hoary rock-rose), Echinacea angustifolia (narrow-leaved purple coneflower), Echinacea purpurea (purple coneflower), Galphimia glauca, Hedera helix (ivy), Melia toosendan (Chinese elderberries, Chin.: Chuan Lian Zi), Olea europaea (olive), Pelargonium sp. (pelargonia), Phytolacca americana (pokeweed), Primula veris (cowslip), Salix sp. (willow), Thymus L. (thyme) Vitex agnus castus (chasteberry), Vitis vinifera (common grape vine), Rumicis herba (sorrel herb), Verbena officinalis (verbena), Sambucus nigra (black elder), Gentiana lutea (yellow gentian), Cannabis sativa (hemp), Silybum marianum (milk thistle).
